(19) United States
(12) Patent Application Publication
(10) Pub. No.: US 2015/0148728 A1
(43) Pub. Date: May 28, 2015

(54) ISOLATED ORTHOSIS FOR THUMB ACTUATION

(71) Applicants: Hani M. Sallum, Somerville, MA (US); Leia Stirling, Stoneham, MA (US); Annette Correia, Milton, MA (US)

(72) Inventors: Hani M. Sallum, Somerville, MA (US); Leia Stirling, Stoneham, MA (US); Annette Correia, Milton, MA (US)

(73) Assignees: CHILDREN'S MEDICAL CENTER CORPORATION, Boston, MA (US); PRESIDENT AND FELLOWS OF HARVARD COLLEGE, Cambridge, MA (US)

(21) Appl. No.: 14/343,458

(22) PCT Filed: Sep. 10, 2012
(86) PCT No.: PCT/US2012/054453

§ 371 Date: Jun. 25, 2014

Related U.S. Application Data
(60) Provisional application No. 61/532,181, filed on Sep. 8, 2011.

(51) Int. Cl. A61F 5/01

(52) U.S. Cl. CPC A61F 5/013
USPC 602/22

(57) ABSTRACT

An orthosis system includes an orthotic device adapted to be worn on the hand of a subject that includes at least one brace component coupled to one or more fingers of the hand and including at least one joint permitting movement of one or more fingers. One or more actuators can be connected to each joint to cause movement of the joint. A control unit can be provided to control each of the actuators to control the movements of each joint separately. The control unit can be operated by the subject or a clinician to facilitate everyday tasks or for treatment or therapy.

### CROSS-REFERENCE TO RELATED APPLICATIONS

This application claims any and all benefits as provided by law, including benefit under 35 U.S.C. §119(e) of U.S. Provisional Application No. 61/532,181 filed Sep. 8, 2011, which is hereby incorporated by reference in its entirety.

### STATEMENT REGARDING FEDERALLY SPONSORED RESEARCH

Not Applicable

### REFERENCE TO MICROFICHE APPENDIX

Not Applicable

### BACKGROUND

1. Technical Field of the Invention

The present invention relates to an actively controlled orthotic device to assist in motion and rehabilitation of a digit, including, for example the thumb.

2. Description of the Prior Art

Fine motor control leading to precision grasping and object manipulation is derived from the human's distinctive opposable thumb morphology. This unique characteristic of the human is aligned with the development and usage of tools (Susman, 1994, Napier, 1962). Many Activities of Daily Living (ADL) involve precision grasping and manipulation, such as brushing one's teeth or feeding oneself. The hand itself is quite complex, containing 27 bones and allowing for numerous kinematic orientations. From birth infants begin with simple grasps and develop finer motor skills with age. The Erhardt Developmental Prehension Assessment (Erhardt, 1994) describes that around 4 months of age the infant will develop a primitive squeeze grasp, where the “hand pulls the object back to squeeze precariously against [the] other hand or body” with no thumb involvement. At 5 months, the infant can use the palmar gasp, where the object is held with fingers and adducted thumb. Starting at 7 months the infant will grasp with an opposed thumb and straight wrist, allowing for the ability to pinch and grasp objects. However, many children with cerebral palsy, stroke, or traumatic brain injury may lose the ability to actively and accurately control the thumb, more precisely their carpometacarpal (CMC) abduction and metacarpophalangeal (MCP) extension, instead having their thumb adducted and flexed in the palm. This pathology makes difficult, or can even prevent, children from grasping with their thumb and fingers, leading to the loss of the more advanced grasps and the ability to easily care for themselves.

Current physical exams by the occupational therapist include evaluating passive and active range of motion of the joints, the fixed and dynamic muscle contractures, and ability to perform common upper extremity tasks. Depending on the severity, treatment can range from orthopaedic surgery, to drugs, to using passive orthotics, or a combination thereof. All treatments are coupled with rehabilitation exercises to improve hand functionality. While not as common, there exist powered grasp assist devices on the market. However, these orthoses generally immobilize the thumb entirely in a neutral position (Broadened Horizons Electric Powered Prehension Orthosis, JAECO Orthopedic Power Driven Flexor Hinge Hand Orthosis). While these devices allow the user to perform grasping actions, there is little opportunity for rehabilitation of the thumb.

Recent work has focused on developing active robotic systems that specifically focus on hand and finger functionality (Dovat et al., 2008; Connelly et al., 2009; Ochoa and Kamper, 2009; Schabowsky et al., 2010). For example, Dovat et al. (2008) have developed a table based system where the finger tips slide into cable loops that are controlled through a motor and pulley system. However, this method of endpoint control can lead to inaccurate joint kinematics as the human arm and hand have many degrees of freedom. Thus, there are different joint orientations that can create the same endpoint positions (Asada and Slotine, 1986). Schabowsky et al. (2010) have improved on the table based system by developing an exoskeleton device that assists motion of the fingers and thumb. However, it does not have a human machine interface component and is designed as a repetitive task motion machine, thus cannot be used for assisting with common tasks required for activities of daily life. While a more portable pneumatic (Connelly et al., 2009) and cable driven system (Ochoa and Kamper, 2009) has the ability to assist with finger extension in a non constrained environment, they do not assist thumb motion.

While sensorimotor rehabilitation of the thumb is an active effort in Physical and Occupational Therapy, there are few tools available for quantitatively analyzing the range of motion of the patient from session to session, making it difficult to gauge overall rehabilitation progress. The most prevalent clinical method for determining range of motion of the thumb is through the use of goniometers. These devices provide a single degree of freedom measure that can be aligned and reoriented on key land marks in order to obtain joint range of motion. For example, it is used for the thumb obtain information on the abduction and flexion. Within a physical therapy session intra-observer reliability is high; however, there is low inter-observer reliability within a session and low reliability between sessions (Elveru et al., 1998; Menadue et al., 2006). These inconsistencies may be caused by inconsistent landmark identification, differences in operator applied torque, and muscle relaxation of the patient (Weaver, 2001). While these devices are a simple to use clinical tool, their use as a means to monitor rehabilitation progress across sessions is limited and they cannot be used during rehabilitation exercises to record the actual motions performed. The current invention has the ability to provide continuous quantitative input on the orientation of the thumb during the rehabilitation session.

In accordance with the invention, the control unit can record movement or motion by recording the distance and direction a pivot joint is moved or by recording a first position of the pivot joint and a second position of the pivot joint. Thus, the recording can include a list of one or more movement directions over a distance or a time period or a series of positional points (e.g., angles) sensed by the sensors.

Research has shown that plasticity exists in the corticospinal system permitting activity dependent neuronal connections to develop (Johansson, 2000; Martin et al., 2007). This experience based remodeling is fundamental for developing rehabilitation programs. For example, constraint induced movement therapy shows that repetitive practice can lead to improved functionality in an adult (Wolf et al., 2008) and pediatric population (Eliasson et al., 2005; Taub et al. 2007). Similarly, work in the field of rehabilitation robotics has shown improvements in gross upper extremity movement with repeated practice using devices that passively relieve the weight of the arm on reaching tasks (Volpe et al., 2000; Houseman et al., 2009).

### REFERENCES

The following references are hereby incorporated by reference in their entirety.
Weaver K, Price R, Czerniecki J, Sangeorzan B. Design and validation of an instrument package designed to increase the reliability of ankle range of motion measurements. Journal of Rehabilitation Research and Development 28(5): 471-475, 2001.
Elveru R, Rothstein J, Lamb R. Goniometric reliability in a clinical setting. Subtalar and Ankle Joint Measurements. Physical Therapy 68:672-677, 1988.
Menadue C, Raymond J, Kilbreath S, Refshauge K, Adams R. Reliability of Two Goniometric Methods of Measuring Active Inversion and Eversion Range of Motion at the Ankle. BMC Musculoskeletal Disorders 7:60, 2006.
Dovat L, Lambercy O, Gassert R, Maeder T, Milner T, Leong T, Burdet E. HandCARE: A cable actuated rehabilitation system to train hand function after stroke. IEEE Transactions on Neural Systems and Rehabilitation Engineering 16(6): 582-591, 2008.
Connelly L, Stoykov M, Jia Y, Toro M, Kenyon R, Kamper D. Use of a pneumatic glove for hand rehabilitation following stroke. In Proceedings for the 31st Annual International Conference of the IEEE EMBS, Minneapolis, Minn., 2-6 Sep., 2009.
Ochoa J, Kamper D. Development of an actuated cable orthotic glove to provide assistance of finger extension to stroke survivors, Journal of Biomedical Engineering 3(5): 75-82, 2009.
Schabowsky C, Godfrey S, Holley R, Lum P. Development and pilot testing of HEXORR: Hand EXOskeleton Rehabilitation Robot. Journal of NeuroEngineering and Rehabilitation 7: 36, 2010.
Asada and Slotine. Robot Analysis and Control. John Wiley and Sons, New York, 1986.
Broadened Horizons Electric Powered Prehension Orthosis (EPPO), http://www.broadenedhorizons.com/powergrip.htm
Erhardt R (1994). Developmental Hand Dysfunction: Theory, Assessment, and Treatment. Published by Pro-Ed, Inc., Austin, Tex.
JAECO Orthopedic Power Driven Flexor Hinge Hand Orthosis, http://jaecoorthopedic.com/products/categories/Wrist-%26-Hand-Orthosis/
Napier J (1962). The evolution of the hand. Scientific American 207:56-62.
Susman R (1994). Fossil Evidence for Early Hominid Tool Use. Science 265(5178):1570-1573.
Johansson B. Brain plasticity and stroke rehabilitation: The Willis lecture. Stroke 31:223 230, 2000.
Martin J, Friel K, Salimi I, Chakrabarty S. Activity and use dependent plasticity of the developing corticospinal system. Neuroscience and Biobehavioral Reviews 31:1125 1135, 2007.
Eliasson A, Krumlinde Sundholm L, Shaw K, Wang C. Effects of constraint induced movement therapy in young children with hemiplegic cerebral palsy: an adapted model. Developmental Medicine and Child Neurology, 47(4):266-75, 2005.
Taub E, Griffin A, Nick J, Gammons K, Uswatte G, Law C. Pediatric CI therapy for stroke induced hemiparesis in young children. Developmental Neurorehabilitation 10(1): 3-18, 2007.
Volpe B, Krebs H, Hogan N, Edelstein L, Diels C, Aisen M. A novel approach to stroke rehabilitation. Neurology 54: 1938-1944, 2000.
Houseman S, Scott K, Reinkensmeyer D. A randomized controlled trial of gravity supported, computer enhanced arm exercise for individuals with severe hemiparesis. Neurorehabilitation and Neural Repair 23 (5): 505-514, 2009.

### SUMMARY

To address the deficiencies of typical orthotic devices and the limitations of clinical methods, the present invention is directed to an isolated orthosis system that can assist in sensorimotor rehabilitation of the digits or fingers of the hand and can be used as an assistive device during every day activities. The orthosis system generally includes an orthotic device designed to be worn on the hand and one or more brace elements, controlled by actuators that are adapted to engage and actuate one or more fingers of the hand. A controller can be provided to control the actuators that manipulate the brace causing the digits to move.

In accordance with one embodiment, a two degree of freedom, Isolated Orthosis for Thumb Actuation (IOTA) is described. This system according to this embodiment can be used to aid in sensorimotor rehabilitation of the thumb, focusing on the carpometacarpal (CMC) and metacarpophalangeal (MCP) thumb joints.

When used as a rehabilitation device, the system according to one embodiment of the invention can be implemented to augment and extend standard occupational therapy. The device can be worn during a standard therapy session to assist with activities, or as an activity in itself. The device can also be used at home to practice tasks introduced during therapy. With continued usage, this device could lead to motor memory that will train the user to use the thumb without need for the device. With permanent additional functionality, the user will have an easier time stabilizing objects and will be able to perform bimanual tasks.

In accordance with one embodiment, the IOTA can be used as an assistive device during every day activities. In this manner, the device can be worn as often as desired to assist in common every day activities, such as putting toothpaste on a toothbrush, feeding oneself, putting on pants, taking money out of a wallet, etc.

In one embodiment, the IOTA can include a semi-rigid hand brace which allows for unrestricted motion of the fingers. A jointed structure mounts around the thumb and the back of the hand. The mounting is adjustable so that the mechanism can be fitted to the individual user. The mounting is also removable for ease of donning the underlying orthosis. The thumb attachment scheme and overall mechanism is designed to minimize the amount of hardware in the palmar region of the hand to prevent obstruction of grasping motions. The IOTA limits the CMC joint to have a single axis of motion, allowing for abduction assist with an opposed thumb grasp. The degree of thumb circumduction can be adjustable to allow opposition grasp, but allow for differences in subject pathology. The MCP joint is assisted in flexion. The interphalangeal (IP) joint is nominally immobilized.

In accordance with one embodiment of the invention, the CMC and MCP joints can be assisted by the IOTA, with each joint independently controllable. In this embodiment CMC adduction is provided by the subject, though adduction rate can be limited by the IOTA. In this embodiment, the CMC and MCP joint angles can be measured directly at the joint structure by optical encoders or other sensors, which are connected by a multi-conductor cable to a control box, which can provide power and read information (e.g., angular position) from the encoders. In this embodiment, the CMC and MCP joints are driven via a flexible cable transmission by two small servo motors, which are contained in the control box.

In accordance with another embodiment of the invention, the control box can be worn in a pack on the patient's upper arm or be placed on any nearby surface. A multi-conductor cable can be used to connect the actuators (e.g., servo packs) to a control box.

In accordance with another embodiment of the invention, the CMC adduction and MCP flexion components can be actively controlled through additional motors.

In a further embodiment, the actuation may be provided by other methods as an alternative to or in addition to servo motors, including but not limited to DC motors, pneumatic actuators, shape memory alloy, traditional electromagnetic devices (e.g., rotary motors and linear actuators), conductive polymers, electroactive polymers, electrostatic devices, or any combination thereof. When actuated by the control system, these methods would convert potential energy (i.e., electrical, compressed gas, fluid pressure, etc.) as supplied by the power source into mechanical energy.

In some embodiments, additional sensors can be incorporated into the system for measuring physical parameters about the system to be used by the control system and/or the control system algorithms. These sensors can include, but are not limited to surface electromyography, accelerometers, gyroscopes, magnetometers, strain sensors, optical sensors, bend sensors, load cells, piezo-resistive sensors, or any combination thereof.

In accordance with some embodiments of the invention, the sensors can produce signals that are input into the control box. The control box can process these external sensor signals and include a program that produces one or more control signals that cause the actuators to actuate one or more of the pivot mechanisms causing the thumb to move. Where the sensors are surface electromyography sensors, attempts by the subject to move the muscles in the hand can be used to drive the actuators and assist the thumb movement.

In accordance with some embodiments of the invention, the orthotic device can be controlled by a self-contained rechargeable control box, which can be operated by the subject or a clinical professional, such as an occupational therapist. The range of motion (ROM) and joint speed for each joint can be set by the operator at any time during operation, however for safety, joint motion can be disabled while settings are being modified.

Further embodiments provide a method for operating the orthotic device in accordance with the invention. The method can include: receiving information indicating an orientation (or a motion, speed and distance) of the CMC and/or the MCP joint and in response to receiving the information, sending one or more actuation signals to at least one active component incorporated within the device. In response to the actuation signal, the at least one active component changes state and causes the orthotic device to adjust the thumb orientation. The system can be used in several modes in accordance with the invention. One mode is a manual operation, where the joint angles can be controlled through the control box or via a remote input by the user or clinician. Another mode of operation is through automatic playback, where one or more pre-recorded motions can be executed. These pre-recorded motions can be developed in collaboration with occupational therapists and hand surgeons in order to follow recommended motion pathways. A further mode of operation includes the use of additional sensors within the system. These sensors may make use of current muscle activity, arm kinematics (that is the arm, wrist, thumb orientations, velocities, or accelerations) in order to select the appropriate actuation signal.

In one operating mode the system can learn a motion profile by providing little or no resistance to the thumb while it is passively moved by the clinician or users through a range of motion. During the passive motion the device can record the motion profile including the joint positions, motions and velocities. This motion profile can be saved into memory for later use. When necessary the saved motion profile can be loaded from memory and executed. The controller executes the motion profile to command the actuators to repeat the motion defined by the profile so that the actively controlled motion matches the previously recorded passive motion.

The orthotic system can be implemented during clinical rehabilitation sessions, during clinical evaluation sessions, during at home rehabilitation exercises, or during activities of everyday life.

These and other capabilities of the invention, along with the invention itself, will be more fully understood after a review of the following figures, detailed description, and claims.

### BRIEF DESCRIPTION OF THE FIGURES

FIG. 1A shows a diagrammatic view of an orthosis system according to one embodiment of the invention.

FIGS. 1B and 1C show diagrammatic views of the orthotic device according to one embodiment of the invention.

FIGS. 2A and 2B show diagrammatic views of the actuation of the MCP joint in an orthotic device according to one embodiment of the invention.

FIGS. 3A and 3B show diagrammatic views of the actuation of the CMC joint in an orthotic device according to one embodiment of the invention shown in FIG. 1.

FIGS. 4A and 4B show diagrammatic views of the actuation of the MCP joint and the CMC joint in an orthotic device according to one embodiment of the invention shown in FIG. 1.

FIGS. 5A-5E show a diagrammatic view of an orthosis system according to an alternative embodiment of the invention.

FIGS. 6A-6B show a diagrammatic view of an orthosis system according to a further embodiment of the invention.

### DETAILED DESCRIPTION OF PREFERRED EMBODIMENTS

The present invention is directed to an isolated orthosis system that can assist in sensorimotor rehabilitation of the digits or fingers of the hand and can be used as an assistive device during every day activities. The orthosis system can generally include an orthotic device designed to be worn on the hand and one or more brace elements, controlled by actuators that are adapted to engage and actuate one or more fingers of the hand. A controller can be provided to control the actuators that manipulate the brace(s) causing one or more digits to move. The controller can include one or more user interface elements, such as a button, a switch, a joystick, a dial or a knob to manually control the motion of the actuator. One or more of the user interface elements can also be embodied in a touch screen based user interface. The orthosis system can also include sensors that can be used to control the motion of one or more actuators in the system (including, but are not limited to, surface electromyography, accelerometers, gyroscopes, magnetometers, strain sensors, optical sensors, bend sensors, load cells, piezoresistive sensors, or any combination thereof). The controller can include one more processors and associated memories that can execute programs stored in one or more memories to cause the actuation of the actuators.

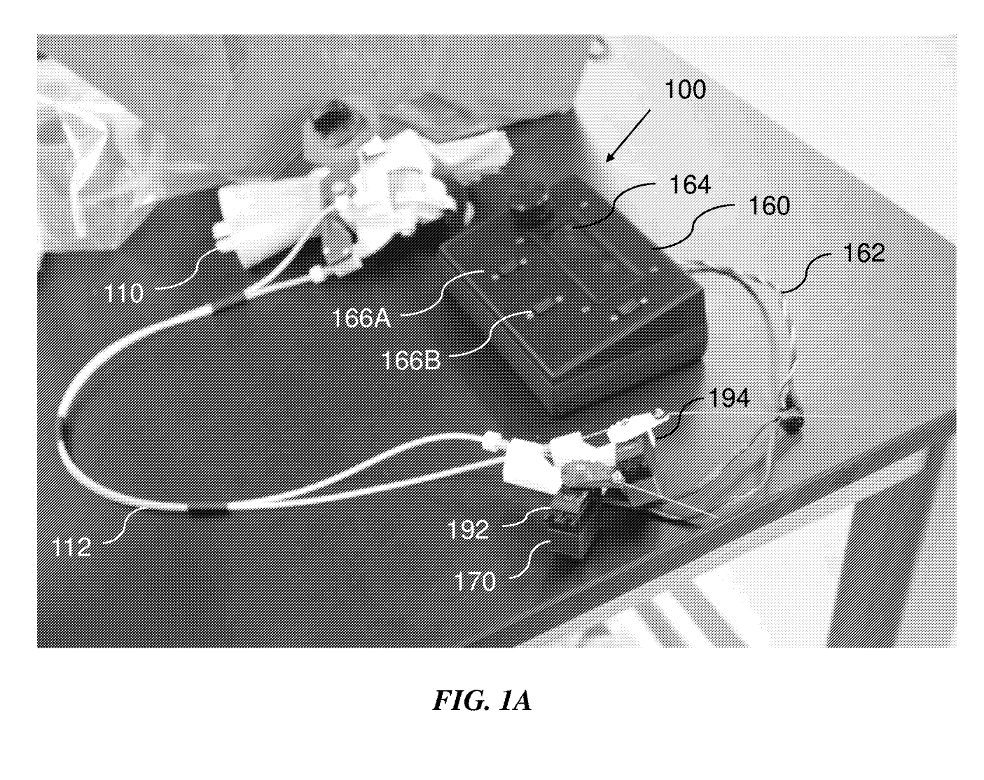

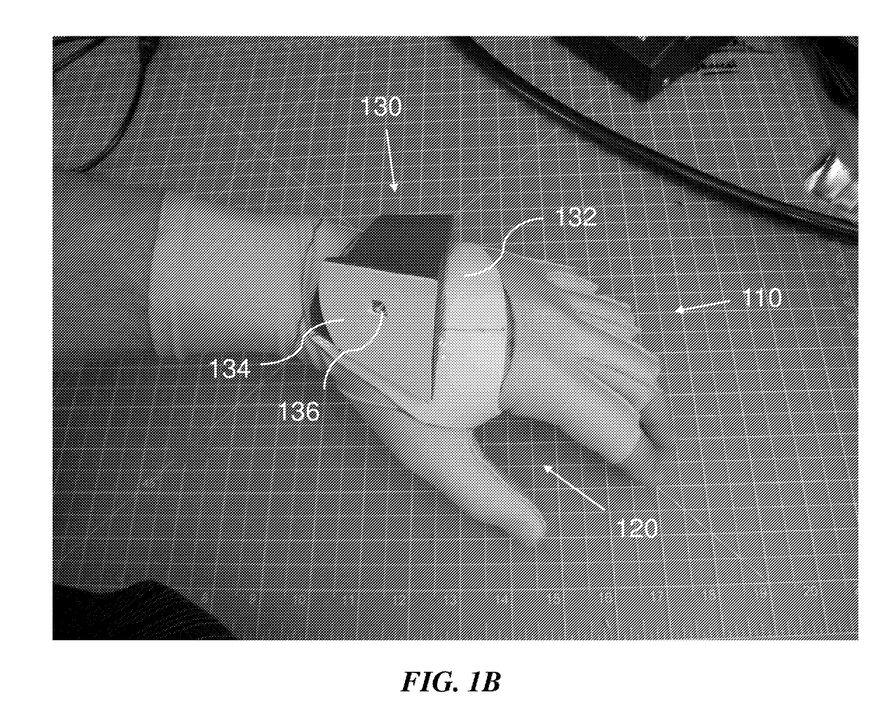

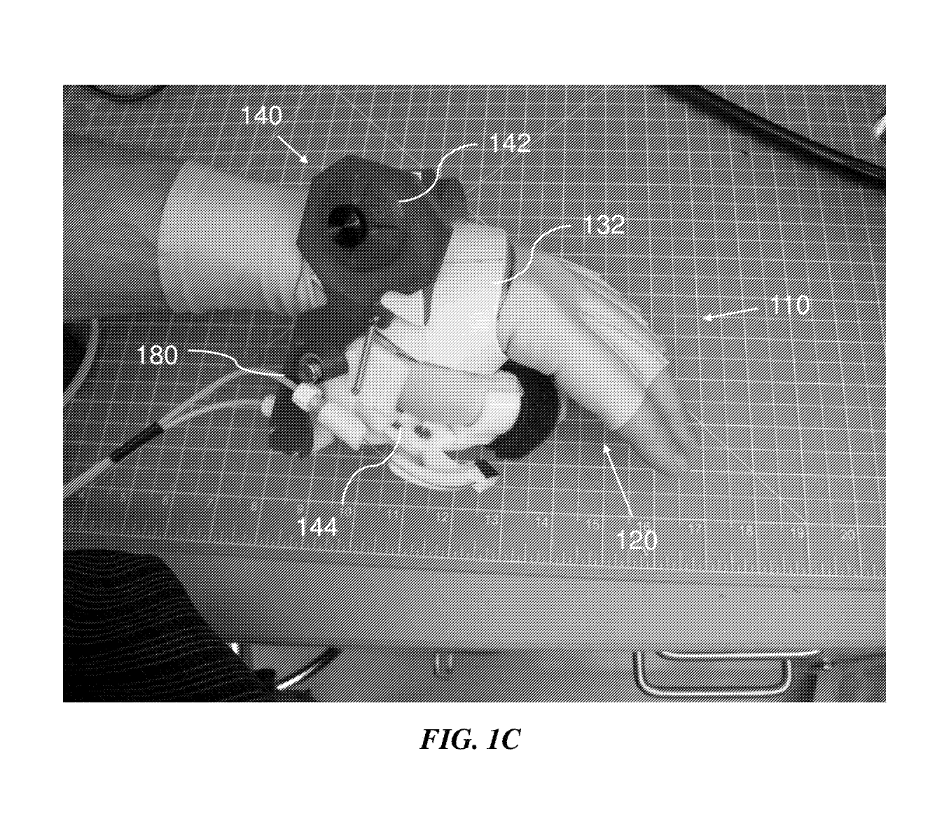

FIG. 1A shows one embodiment of an isolated orthosis system 100 according to the invention. The orthosis system 100 can include an orthotic device 110 connected to guide wires 112 controlled by one or more actuators 170 and a control unit 160. In some embodiments, the actuators can be located inside the control unit 160. Joint angle sensors 192 and 194 on the orthotic device can be connected to the control unit 160 by wires 162. The control unit 160 can be connected to each actuator 170 by wires 162 to control each actuator 170. The control unit can include a display panel 164 and toggle or rocker switches 166A, 166B to enable a user to control the motion of each actuator 170.

FIGS. 1B and 1C show diagrammatic view of the orthotic device 110 according to one embodiment of the invention. In accordance with one embodiment, the orthotic device 110 can include a support element 120, such as a wearable glove, a mounting portion 130 and a brace portion 140. FIG. 1B shows the orthotic device 110 with the brace portion 140 removed for clarity. As shown in FIG. 1B, the mounting portion 130 can include a mounting plate 132 securely fastened to the glove 120 by adhesive, stitching, or any other fastening method. In this embodiment, the mounting plate 132 can include a sheet of substantially rigid plastic material, such as nylon, Delrin, PVC, ABS, that can be molded or formed fit against a portion of the hand. The mounting plate 132 can be formed to wrap around a portion of the hand to provide better stability.

In one embodiment of the invention, the mounting portion 130 can also include a mounting bracket or mounting block 134 that can be securely fastened to the mounting plate 132 by an adhesive or fasteners. The mounting bracket or mounting block 134 can include mounting hole 136 or other mounting element that can be used to mount the brace portion 140 to the orthotic device 110. In one embodiment, the mounting hole 136 can be threaded to enable the brace portion to be bolted to the mounting block 134.

The brace portion 140 can include a plate 142 that can be securely fastened by a bolt to threaded hole 136 in mounting block 134.

In another embodiment of the invention, the brace portion can contain a mounting portion that can be securely fastened to the underlying brace using an adhesive or bonding material. The mounting portion can be a flexible material that can be molded by an occupational therapist (including, but not limited to thermoplastic, flexible aluminum, etc). The moldable portion can be adjusted to the nominal curvature of the hand. Tabs on each end of the moldable portion can then be manipulated to provide the required support of the user. The tabs can be shortened as required by the occupational therapist. The moldable portion can include a railing for the brace portion to connect to.

The brace portion can contain a spring-loaded tensioner that attaches to the railing. This tensioner can include alignment pins.

The brace portion 140 can further include a finger brace 144 adapted to engage one or more fingers, for example, as shown in FIGS. 1B and 1C, the thumb. One or more straps, Velcro™ or elastic bands can be used to secure the finger to the finger brace 144. The finger brace 144 can be connected to the plate 142 by one or more pivot joints or mechanisms 150, 180. Each pivot mechanism 150 or 180 can include a pin that enables at least a portion of the finger brace to pivot relative to the plate 142 or can include addition components, such as bearings or bushings to facilitate rotation. The plate 142 can include a guide wire cable connection that connects to the sheath encasing the guide wire and the finger brace 144 can include a cable end capture component that connects to the end of the guide wire cable, such that movement of the guide wire 112 causes the finger brace 144 and the attached thumb to move.

In one embodiment, the finger brace 144 can be connected to the plate 142 by a spring that biases the finger brace 144 into a predetermined position and the cable end capture component can capture the guide wire cable end in only one direction, e.g. pulling the guide wire cable moves the finger brace 144, while pushing the guide wire cable does not apply any force on the finger brace 144. In this embodiment, the spring serves to bias the finger brace in one direction while allowing for compliant motion of the finger brace 144 and the attached finger.

In this embodiment, the brace portion 140 enables the finger brace 144 to pivot about two pivot points, one, the CMC pivot 180, approximating the CMC joint of the hand and the other, the MCP pivot 150, approximating the MCP joint of the hand. This enables rehabilitative therapy in two degrees of freedom.

In accordance with one embodiment, the finger brace 144 can include a pivot joint 180 (herein referred to the CMC joint 180) for moving the thumb about the CMC joint of the hand and a pivot joint 150 (herein referred to as the MCP joint 150) for moving the thumb about the MCP joint of the hand. The motion of these two joints can be combined and coordinated to improve sensorimotor function.

In accordance with one embodiment of the invention, the CMC joint 180 and MCP joint 150 can be assisted by operation of the control unit 160 and each joint can be independently controlled from the other joint. In this embodiment of the invention, CMC adduction can be provided by the subject, though an adduction rate control that can be limited by the orthosis system 100. In this embodiment of the invention, MCP flexion can be provided by the subject, though a flexion rate control that can be limited by the orthosis system 100. In other embodiments, CMC abduction and/or MCP extension can be provided by the subject through an abduction and/or extension rate control that can be limited by the orthosis system 100. In this embodiment of the invention, the CMC 180 and MCP 150 joints can be driven separately via individual flexible transmission cables 112 controlled by two small servo motors 172, 174, integrated into the control box 160. In an alternate embodiment, the CMC 146 and MCP 148 joints can be driven separately via individual flexible transmission cables 112 by two small servo motors located apart from the control unit 160 in a pack which can be worn on the user's arm, waist belt, or placed on any nearby surface. A multi-conductor cable 162 or wire connection can connect the servo motors 172, 174 to the control unit 160.

In alternative embodiments, additional motors or actuators can be provided such that a separate motor controls CMC abduction, CMC adduction, MCP flexion and MCP extension. In other embodiments, actuators 170 can include DC motors, pneumatic actuators, shape memory alloy, traditional electromagnetic devices (e.g., rotary motors and linear actuators), conductive polymers, electroactive polymers, electrostatic devices, and combinations thereof. Different types of actuators can be used for different motions. When actuated by the control system, these actuators convert potential energy (i.e., electrical, compressed gas, fluid pressure etc.) as supplied by the power source into mechanical energy.

In accordance with one embodiment, the position of the CMC joint 180 or the MCP joint 150 can be determined from sensors included in actuators 170. For example, after initial calibration, the position of a servomotor actuator 170 can be used to determine the position of the joint it controls. Alternatively, one or more of the joints can include a sensor to independently sense and report the position of the joint. In some embodiments, sensors can be provided on the brace portion 140 to monitor and report to the controller, the detected position, velocity, acceleration and forces being experience by the finger brace 144 and the finger. These sensors can include, but are not limited to surface electromyography, accelerometers, gyroscopes, magnetometers, strain sensors, optical sensors, optical encoders, hall-effect sensors, bend sensors, load cells, piezoresistive sensors, or any combination thereof.

The brace 140 can include a sensor that measures the angle of the wrist during use, in order to detect wrist flexing to control thumb motion. This wrist motion can be generated as part of the tenodesis effect of passive finger flexion in response to wrist extension. The wrist motion can also be generated purposefully as an interface modality. In one embodiment, the sensor to monitor wrist flexion is a bend sensor, where the curvature of the sensor is correlated to the wrist angle. The bend sensor spans the wrist joint, such that flexion or extension of the wrist generates a change in resistance of the bend sensor.

In another embodiment of the invention, inertial measurement units (IMUs), comprising accelerometers and gyroscopes can be used to determine wrist angle by incorporating one IMU on the hand and another IMU on the forearm. A wrist angle value can be computed as a function of the relative measure of angle between the two IMUs.

In an alternative embodiment of the invention, Hall Effect or optical sensors can be incorporated to measure wrist angle. In these embodiments, the transducers can be placed on one part of the orthotic that is fastened to one part of the body (e.g., the hand or forearm) and the source (e.g., magnet, light source) can be place on another part of the orthotic that fastened to the other part of the body (e.g., the forearm or hand). As the wrist flexes or extends, the physical relationship (e.g., distance and/or angle) between the transducer (e.g., Hall Effect or optical sensor) and the source (e.g., magnet, light source) changes allowing the flexion angle to be determined as function of the sensor output.

Other embodiments may include wrist angle sensing using surface electromyography, accelerometers, gyroscopes, magnetometers, strain sensors, optical sensors, optical encoders, hall-effect sensors, load cells, piezo-resistive sensors, or any combination thereof.

In accordance with some embodiments, an optical encoder position sensor can be coupled to either CMC joint 180 or MCP joint 150, or both to allow the position of each joint to be determined at the joint location. The motion of actuators 170 can be monitored by encoders at each joint to determine absolute and relative positions of each joint. The slack in the control cables can be determined by moving the joint in one direction and then in the other and determining how much the actuator moves before the encoder records a position change.

The slack in the control cables can be actively controlled by using the encoders at the joint location and at the actuator.

In accordance with one embodiment, the control unit 160 can include a small computer or microprocessor that includes a processor and associated memory and one or more programs that interact with hardware interfaces to control the actuators 170 and move the brace portion 140. The control unit 160 can be battery operated or connected to a power source, either by a wired or wireless connection. The control unit 160 can include programs that are intended to serve therapeutic purposes to treat a subject with disability, or to provide them with an additional assist during everyday life. The control unit 160 can include memory and associated programs to store the motion information of the subject while using the device. The motion information can be used show improvements in range of motion and speed and accuracy of motion. The motion information can also be used to record the motion of the thumb for playback (as therapy) in the future. The control unit 160 can be operated by the subject or a clinical professional, such as an occupational therapist. The range of motion (ROM) and joint speed for each joint can be set by the operator at any time during operation, however for safety, joint motion can be disabled while settings are being modified. The control unit 160 can also record the joint position and speed of motion during operation by the subject so that the motion profile can later be repeated by the device, or reviewed and analyzed by a clinical professional, for example, to design therapeutic exercises or new assistive motions for the subject.

In other embodiments, the control unit 160 can function as a wired or wireless interface (or relay) connected to a remote computer system that provides the control functions described herein. In this embodiment, signals can be sent to the remote computer which is executing an algorithm that determines the appropriate control signals to send back to the control unit 160 to be used to control the actuators 170. The remote computer can be implemented to send high level commands such as turn on, turn off, change mode, etc, in addition to low level actuator-dependent control signals. In embodiments where the remote computer sends high level commands, the low level control signals would be handled locally by the microprocessor. In some embodiments, the functions of the control unit 160 can be incorporated in mobile telephone or similar wireless device. Connections to the sensors and the actuators can be provided through wired connection (e.g. USB) or a wireless connection (e.g. Blue Tooth, Zigbee, WiFi) and connections to the remote computer via a wireless data network (e.g., 3G, 4G, WiFi, WiMAX). In an alternative embodiment, the functions of the control unit 160 can be embedded in a mobile smart phone, table or personal computer executing one or more applications. In these embodiments, the user interface can include switches, buttons, and/or keys or image on a touch screen interface. In addition, voice control can also be provided.

In accordance with other embodiments of the invention, sensors can be used to measure the muscle control signals intended to cause the desired motion of the hand and then cause the corresponding thumb motion by actuating the one or both actuators to assist in the desired motion.

In other embodiments, additional sensors connected to the subject can be used to detect nervous system signals or muscle activity to move one or more of the joints and in response, activate the actuators to cause the joint to move accordingly.

In accordance with one embodiment of the invention, Electromyography (EMG) can be used to measure the signals produced by the skeletal muscles used in the articulation of the thumb.

In one embodiment, the EMG signals can be in direct one-to-one control, that is a specific muscle contracting is directly responsible for determining the motion of the thumb.

In other embodiments, the EMG signals can be recorded from the forearm and algorithms developed (for example, using table look-ups or machine learning methods) to determine which muscle signals correspond to desired motions.

In operation, the subject or a clinical professional can activate one of the switches or controls on the control unit 160 that control a specific joint, for example, a CMC switch activated one way causes the CMC abduction and activated an different way causes CMC adduction or an MCP switch activated one way causes the MCP extension and activated an different way causes MCP flexion. These motions can be combined to improve motor function.

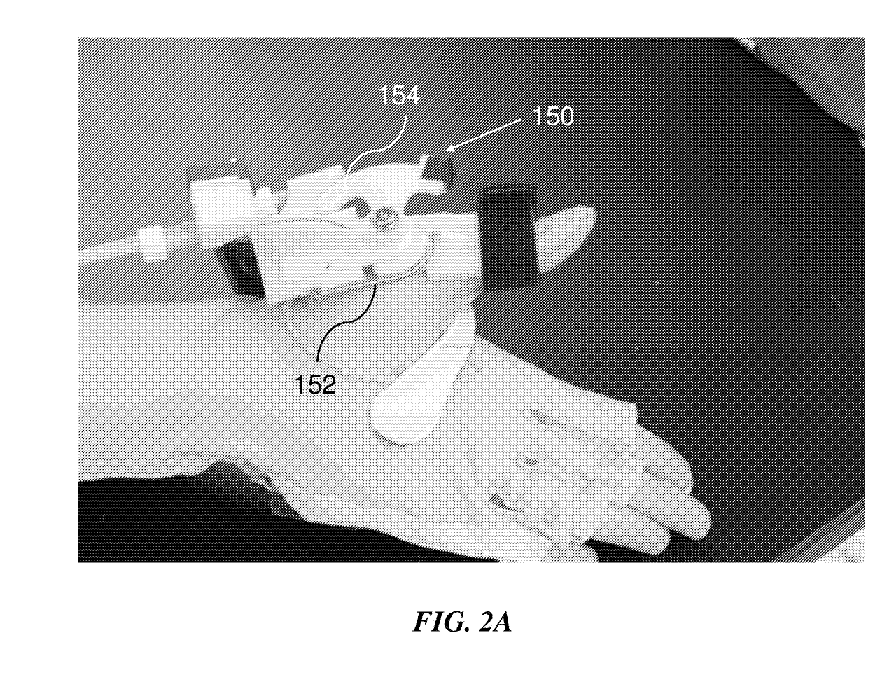

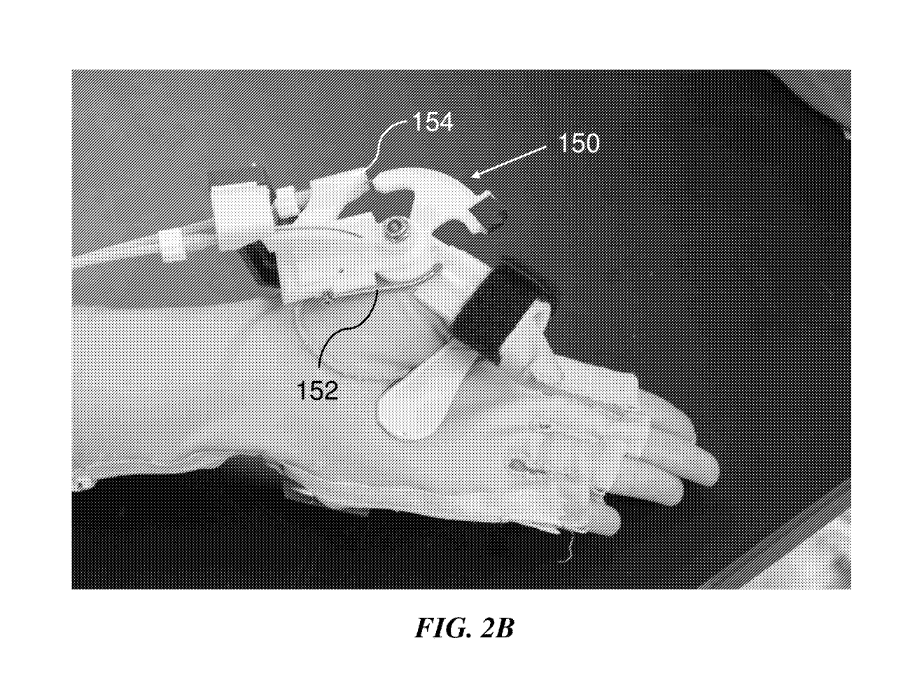

FIGS. 2A and 2B show the flexion joint 150 of the orthotic device engaged in MCP flexion and MCP extension in accordance with one embodiment of the invention. FIG. 2A shows the MCP joint 148 in extension and FIG. 2B shows the MCP joint 148 in flexion. In accordance with one embodiment, the spring 152 biases the MCP joint 148 into flexion and the guide wire cable 154 drives the MCP joint 148 into extension. In other embodiments, a spring (like 152) can be used to bias the MCP joint 148 into extension and the guide wire cable 154 can be used to drive the MCP joint 148 into flexion.

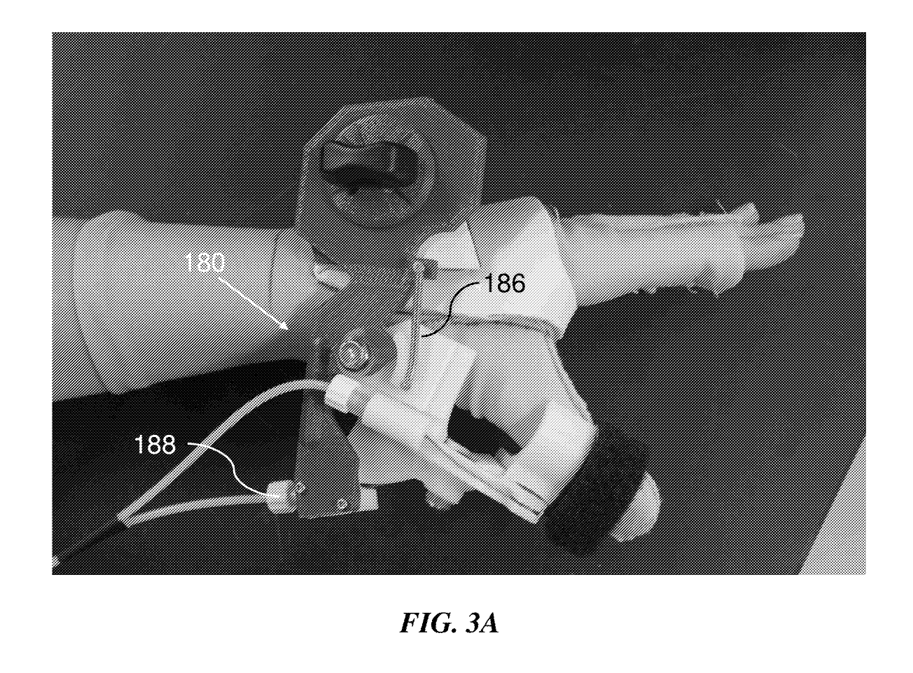

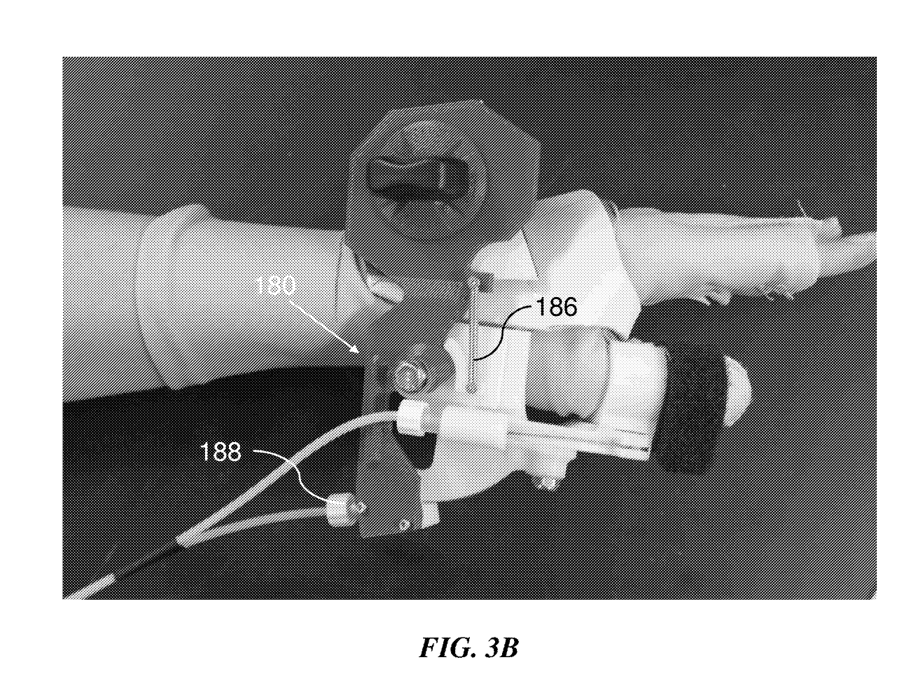

FIGS. 3A and 3B show the orthotic device engaged in CMC abduction and CMC adduction in accordance with one embodiment of the invention.

FIG. 3A shows the CMC joint 146 in abduction and FIG. 3B shows the CMC joint 146 in adduction. In accordance with one embodiment, the spring 156 biases the CMC joint 146 into adduction and the guide wire cable 158 drives the CMC joint 146 into abduction. In other embodiments, a spring (like 156) can be used to bias the CMC joint 146 into abduction and the guide wire cable 158 can be used to drive the CMC joint 146 into adduction.

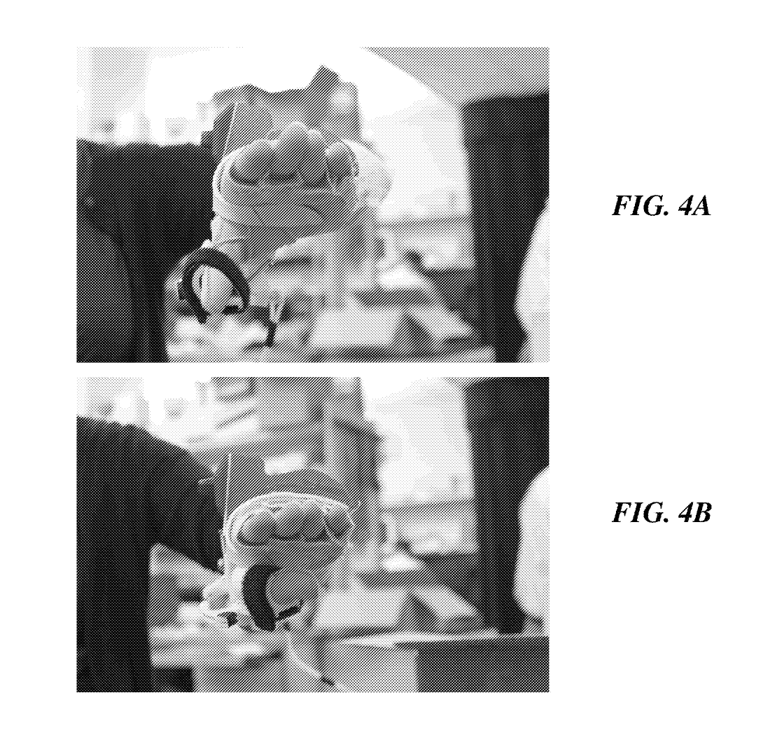

FIGS. 4A and 4B show the orthotic device engaged in both CMC abduction/MCP extension and CMC adduction/MCP flexion in accordance with one embodiment of the invention. FIG. 4A shows the CMC joint 146 in abduction and the MCP joint 148 in flexion at the same time positioning the hand in an open form ready for grasping. FIG. 4B shows the CMC joint 146 in adduction and the MCP joint 148 in flexion at the same time positioning the hand in closed form to grasp an object.

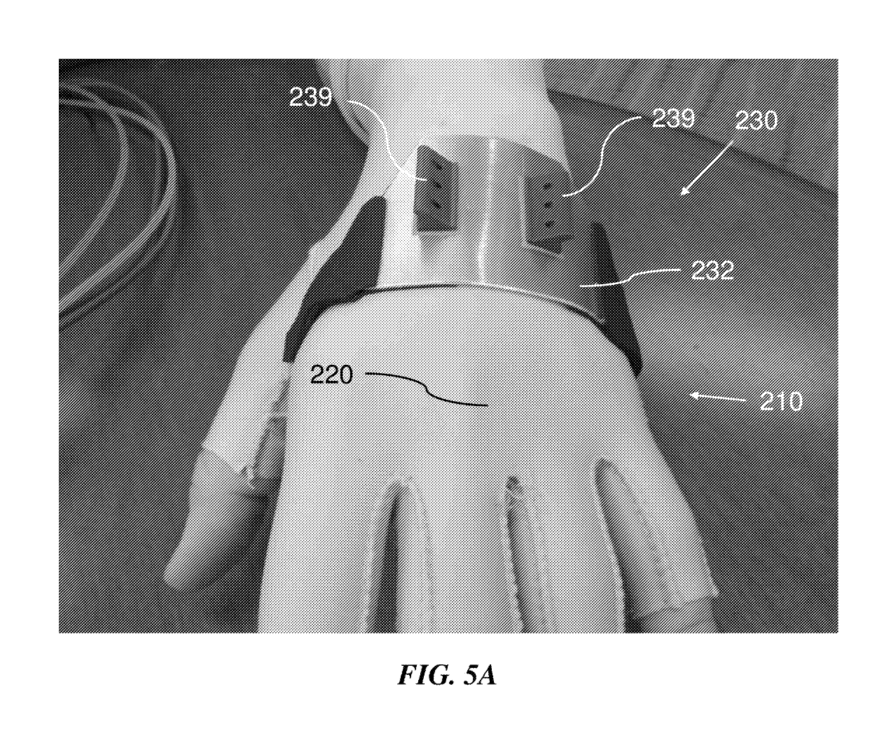

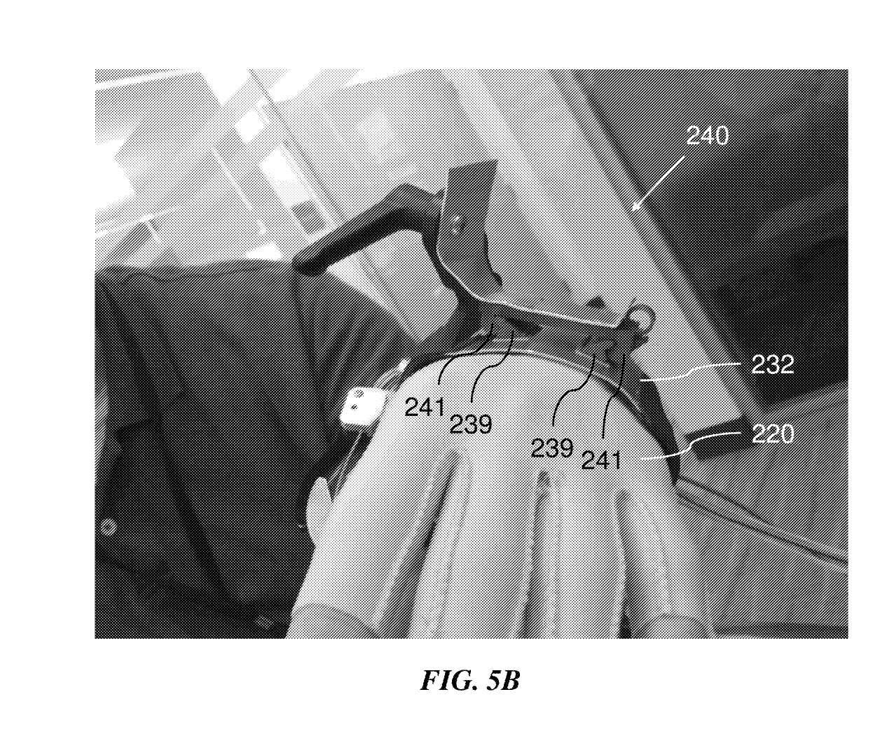

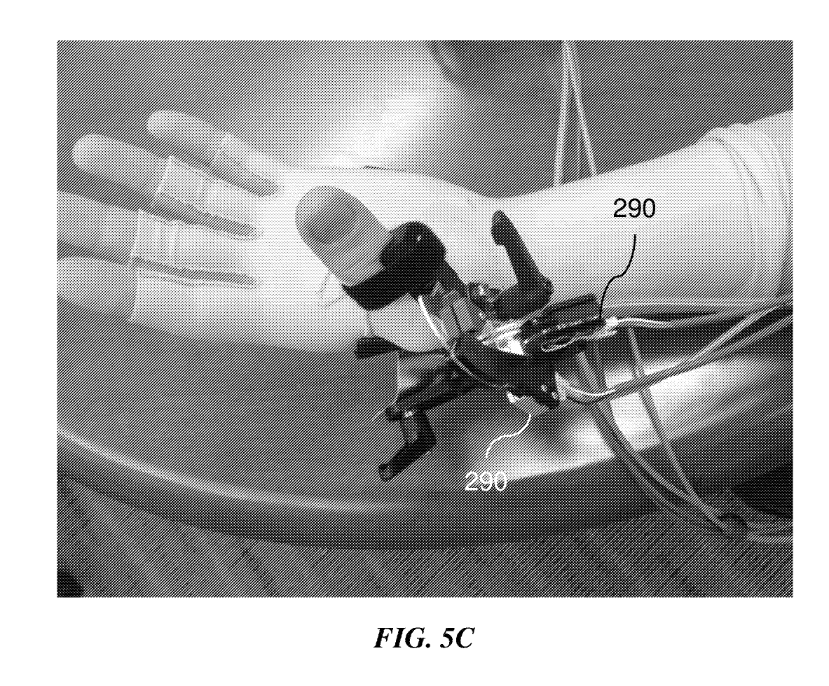

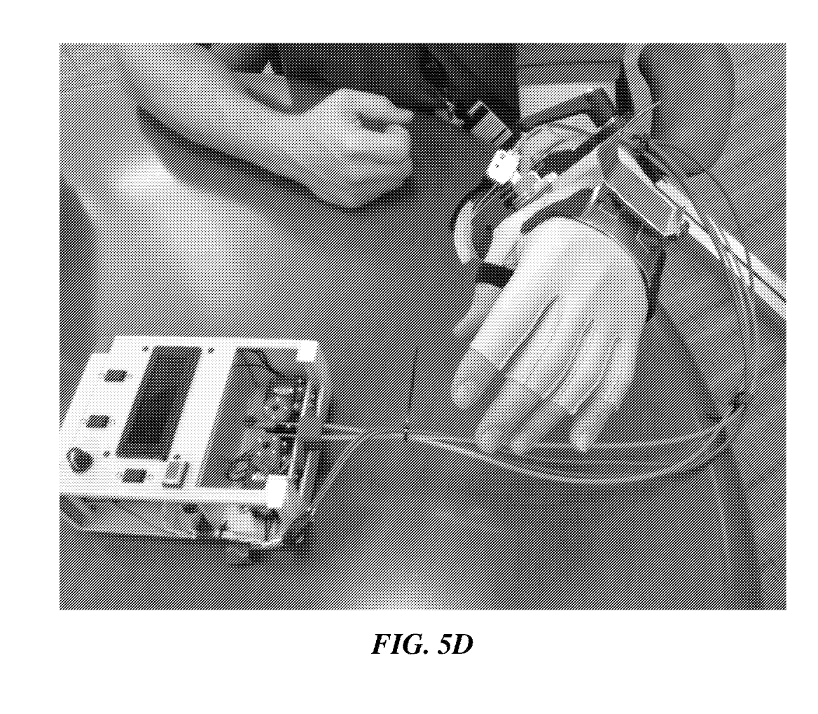

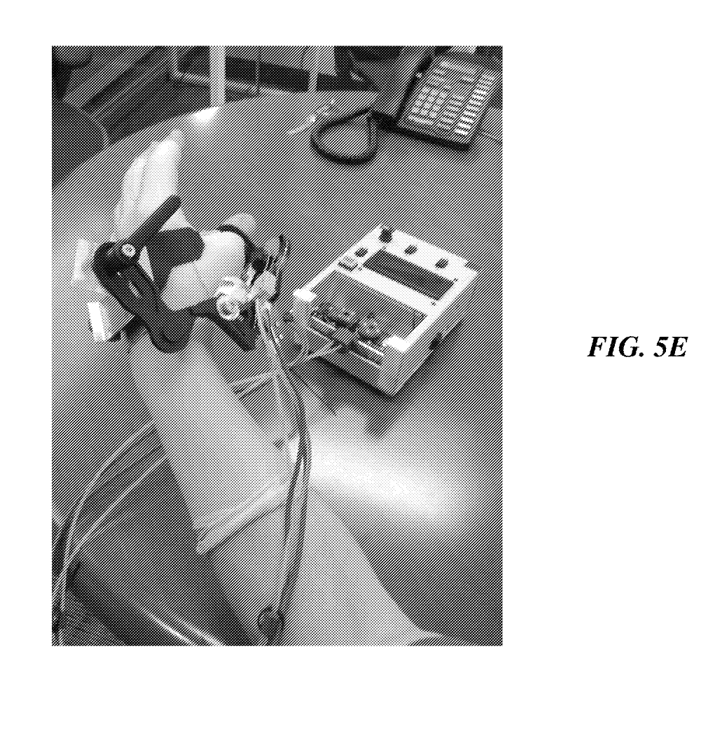

FIGS. 5A-5E show diagrammatic views of the orthosis system 200 according to one embodiment of the invention. In accordance with one embodiment, the orthotic device 210 can include a support element 220, such as a wearable glove, a mounting portion 230 and a brace portion 240. As shown in FIG. 5A, the support element FIG. 5A shows the orthotic device 110 with the brace portion 140 removed for clarity. As shown in FIG. 5A, the mounting portion 230 can be fastened to the support element, glove 220 by adhesive, stitching, or any other fastening method and tabs 235 and 237 can be provided to stabilize the mounting portion 230. The length of tabs 235 and 237 can be extended or shortened to provide stability according to the needs of the subject. The mounting portion 230 can include rails 239 that mate with complementary elements 241 on the brace portion 240 to allow the brace portion 240 to be easily coupled to and positioned on the mounting portion 230. Fasteners or spring clamps can be used to fasten the brace portion 240 to the mounting portion 230. In this embodiment, the mounting plate 232 can include a sheet of substantially rigid material, such as metal, such as steel or aluminum, or a plastic material, such as thermoplastic splinting material, nylon, Delrin, PVC, ABS, that can be molded or formed fit against a portion of the hand. In this embodiment, the mounting plate 232 can be made from a combination of metal and plastic. The mounting plate 232 can be formed to wrap around a portion of the hand to provide better stability. FIG. 5B shows the brace portion 240 mounted on the rails 239 of the mounting portion 230. FIG. 5C shows a side view of the orthotic device 210. In this embodiment, optical encoder sensors 290 can be provided at each of the pivot joints to indicate the angle of orientation of the MCP or CMC joint. FIG. 5D shows a view of the orthosis system 210 according to this embodiment of the invention. FIG. 5E shows a side view of the orthosis system 210 according to this embodiment of the invention.

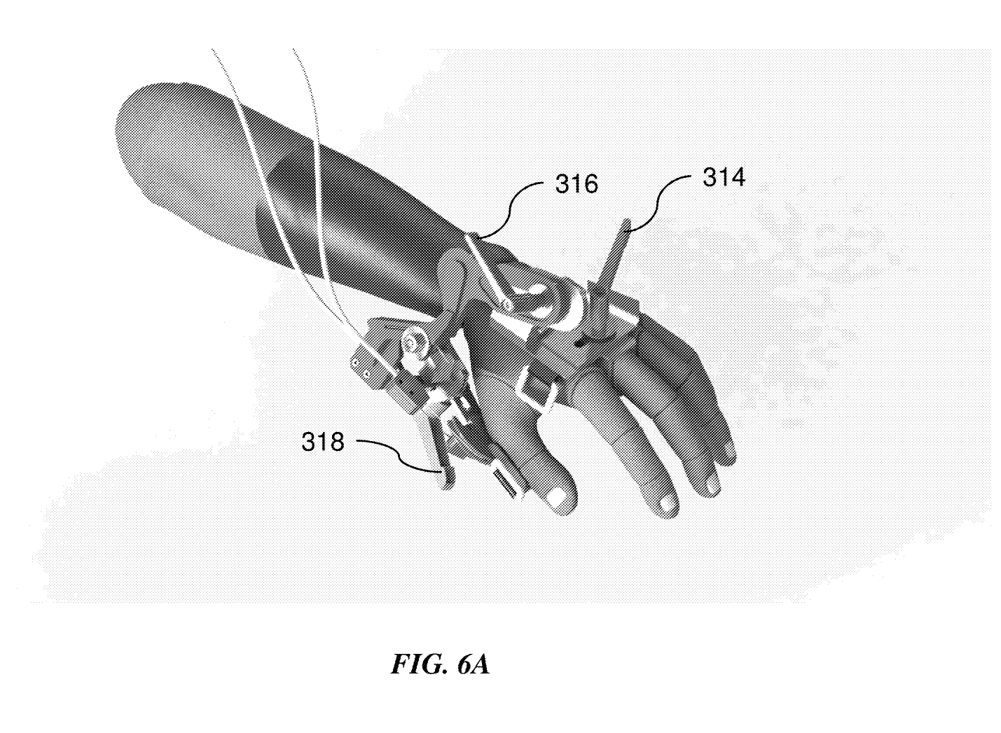

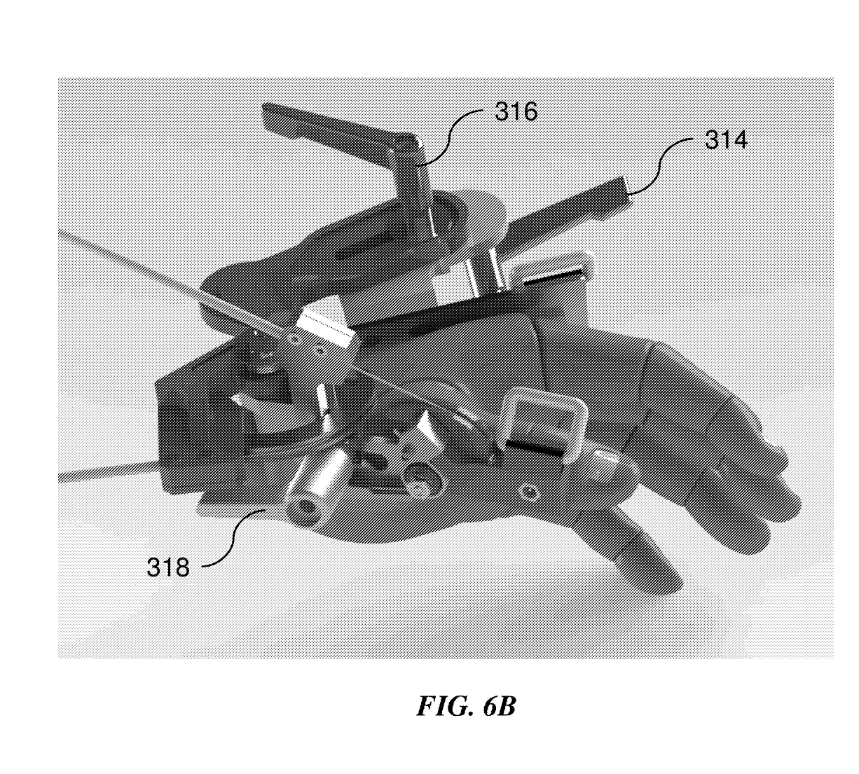

FIGS. 6A and 6B show diagrammatic views of an orthosis system 300 according to an alternative embodiment of the invention. In this embodiment, threaded fasteners 314, 316 and 318 can be used to mount the brace portion to the mounting portion and to enable the pivot elements of each of the joints to be adjusted to the anatomy of the subject.

Further embodiments of the present invention can be used to provide a method for operating the orthotic device 100, 200, 300. The method can include: receiving information indicating the orientation of at least one of the CMC and MCP joint and in response to receiving the information, sending an actuation signal to at least one actuation component 170 of the orthotic device 100. In response to the actuation signal, at least one actuation component 170 changes state and causes the orthotic device to adjust the thumb orientation. In some embodiments, the system can be used in several algorithm based modes. For example, one mode can be a manual operation, where the joint angles are controlled through the control box by the subject or clinician. In another example, a second mode of operation can provide for automatic playback, where a pre-recorded motion, set of motions or exercises can be executed. These pre-recorded motions can be recorded by the device while the user's thumb is passively moved by the clinician or the user. The pre-recorded motions can also be developed in collaboration with occupational therapists and hand surgeons in order to follow recommended motion pathways physical therapy or occupational development. A further mode of operation can include the use of additional sensors connected to the control unit 160 of the system 100. The sensors detect muscle activity, kinematics (e.g., the arm, wrist, thumb, finger orientations, velocities, and/or accelerations), and/or forces and torques. The control unit can operate on these signals and compute the appropriate actuation signal.

Systems according to the various embodiments of the invention can be used as part of clinical rehabilitation sessions, clinical evaluation sessions, or during at home rehabilitation exercises.

Systems according to the various embodiments of the invention can also be used as an assistive device during everyday life.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. An orthotic system comprising:
an orthotic support element adapted to be worn on the hand of a subject, the orthotic device including at least one brace component connected to the support element and coupled to at least one finger of the hand of the subject and including a first pivot joint permitting movement of the at least one finger in a first dimension;
a first actuator connected to the first pivot joint such that actuation of the actuator causes movement of the first pivot joint in the first dimension; and
a control unit connected to the first actuator and providing first control signals to the first actuator to control the actuation of the first actuator.

2. The orthotic system according to claim 1 further comprising a second pivot joint permitting movement of the at least one finger in a second dimension and a second actuator connected to the second pivot joint and the control unit, the control unit providing second control signals to the second actuator to control the actuation of the second pivot joint.

3. The orthotic system according to claim 1 further comprising a first sensor connected to the first actuator for sensing a position of the first pivot joint.

4. The orthotic system according to claim 2 further comprising a second sensor connected to the second actuator for sensing a position of the second pivot joint.

5. The orthotic system according to claim 1 further comprising a first sensor coupled to the first joint for sensing a position of the first pivot joint.

6. The orthotic system according to claim 2 further comprising a second sensor coupled to the second joint for sensing a position of the second pivot joint.

7. The orthotic system according to claim 1 where the first pivot joint aligns with the CMC joint of the thumb.

8. The orthotic system according to claim 1 where the second pivot joint aligns with the MCP joint of the thumb.

9. The orthotic system according to claim 1 wherein the control unit receives input signals and the control unit, as a function of the input signals, outputs a control signal to the first actuator causing the first pivot joint to move in at least one direction.

10. The orthotic system according to claim 1 wherein the control unit is connected to at least one external sensor and the control unit receives external signals from at least one external sensor, and the control unit, as a function of the external signals, outputs a control signal to the first actuator causing the first pivot joint to move in at least one direction

11. The orthotic system according to claim 10 where in the external sensor is a surface electromyography sensor.

12. A method of using an orthotic system comprising:
providing an orthotic support element adapted to be worn on the hand of a subject, the orthotic device including at least one brace component connected to the support element and coupled to at least one finger of the hand of the subject and the at least one brace component including a first pivot joint permitting movement of the at least one finger in a first dimension, the orthotic system also including a first actuator connected to the first pivot joint such that actuation of the actuator causes movement of the first pivot joint in the first dimension and a control unit connected to the first actuator and providing first control signals to the first actuator to control the actuation of the first actuator; the control unit including at least one user interface element to provide input to the control unit;
operating the at least one user interface element to provide input to the control unit whereby the at least one finger is caused in a range of motion according to the first pivot joint.

13. The method according to claim 12 wherein a user operates the at least one user interface element to perform at least one every day activity.

14. The method according to claim 13 wherein the at least one every day activity includes one or more of putting toothpaste on a toothbrush, brushing one's teeth, feeding oneself, putting on clothing, and taking money out of wallet.

15. The method according to claim 12 wherein a care provider operates the at least one user interface element to cause the at least one finger to move over a predefined range of motion, and
recording, by the controller, at least some of the movements over the predefined range of motion; and
playing back at least some of the recorded movements.

16. The method according to claim 12 wherein the orthotic includes at least one sensor on the first pivot joint, and
manually moving the at least one finger over a predefined range of motion;
recording and storing at least some of the movements of the at least one finger over the predefined range of motion; and
playing back at some of the recorded movements.
